(19) United States
(12) Patent Application Publication
(10) Pub. No.: US 2025/0066613 A1
(43) Pub. Date: Feb. 27, 2025

(54) ATROROSIN BASED LAKE

(71) Applicant: CHROMOLOGICS ApS, Søborg (DK)

(72) Inventors: Anders Sebastian Rosenkrans ØDUM, Søborg (DK); Sara THODBERG, Søborg (DK)

(73) Assignee: CHROMOLOGICS ApS, Søborg (DK)

(21) Appl. No.: 18/726,290

(22) PCT Filed: Jan. 5, 2023
(86) PCT No.: PCT/EP2023/050143

§ 371 Date: Jul. 2, 2024

(30) Foreign Application Priority Data
Jan. 5, 2022 (DK) PA202270003

(51) Int. Cl. C09C 3/00; A23L 5/46; C09C 1/40; C09C 3/04; C09C 3/06; C09C 3/10

(52) U.S. Cl. CPC C09C 3/006; A23L 5/46; C09C 1/407; C09C 3/041; C09C 3/06; C09C 3/10; C01P 2004/62

(57) ABSTRACT

A formulation of Atrorosins, and Atrorosin Lakes. Additionally, methods for their production by binding to inert metallic mordant is provided; and further the uses of the lakes as a coloring agent for food items and/or non-food items, and for cosmetics.

### TECHNICAL FIELD OF THE INVENTION

The invention relates to the field of natural food colorants. Specifically the invention relates to the field of formulations of food colorants, lakes of colorants, methods for their production by binding to inert metallic mordant and uses of the novel pigments as a coloring agent for food items and/or non-food items, and for cosmetics.

### BACKGROUND OF THE INVENTION

During the last decade, consumer chemistry has become a focal point of interest. This includes fragrances, flavors, and colorants in food and non-food products. In foods, most companies have already switched from synthetic to natural colorants as they are perceived to be more healthy, safe, and authentic. There is a strong market pull for high performing natural colorants in industry segments such as food, cosmetics, and home care. Red is the colorant with the broadest application range within foods and takes up to 55% of the market share.

Atrorosins are a new class of colorants biosynthesized by Talaromyces atroroseus. Atrorosins, have an azaphilone scaffold and are red colorants which acts as dyes in water as they have a high solubility. Uses of Atrorosins are currently being explored especially in the foods as natural colorants are of high demand however other industries such as cosmetics, coatings, inks, and paints are also looking at alternatives to petroleum-based colorants.

Atrorosins, like many natural dyes, have poor stability to environment, such as photooxidation by UV light, chemical interactions with other compounds and susceptible to hydrolysis in high water activity matrices compared to synthetic dyes. This instability can lead to a change in color tone or discoloration over time which is undesirable. Sometimes these weaknesses can be masked by co-formulation of other additives, or oversaturation of colorant in the product, however these solutions would like to be avoided.

These physiochemical weaknesses can be overcome by stabilizing the natural dye with an inert metallic mordant. These colors are referred to as laq or lakes. The most used mordant is aluminum, but other metallic compounds such as magnesium, iron, calcium have all been proposed. Stabilization of dye with a metallic mordant, will change the dye into a pigment and change the physiochemical properties of the colorant to be much more stable versus the environment such as pH, UV, temperature, and water activity. Increasing the stability against environmental factors and making the Atrorosin lakes water insoluble will give a greater diversity in color toning as milling can help define the tone from dull to bright. However, present methods of making Atrorosin lakes produce lakes that will not stay fully dispersed in liquids, and which form precipitates, and provide lake colors that are not brilliant and not very powerful. The present invention provides solutions to those problems by providing a new method of making Atrorosin lakes that will stay dispersed to a higher degree than seen with previous methods. Further, the new method provides brilliant and powerful colored lakes.

WO2022/008503 discloses scalable methods for producing atrorosins and compositions comprising atrorosins.

### SUMMARY OF THE INVENTION

In view of the preceding, the problem addressed by the present invention is to provide a new subset of Atrorosin pigments, Atrorosin lakes, where the color imparted on the pigments are based on Atrorosins, and where the Atrorosin lakes are dispersible in liquids, such as in water. Atrorosin-(R)—X— where in R is a hydrogen or N—R is selected among, an amino acid, a peptide, an amino-sugar and a primary amine, and X is a metal selected among Aluminum, Copper, Nickel, Cobalt or Zinc. Further, the present invention solves the problem of providing brilliant and powerful lake colors by providing a new and improved method for making brilliant, powerful, stable, and highly dispersible lakes, as well as providing the lakes such as Atrorosin lakes, such as AtrorosinE lakes.

In a first aspect the present invention provides an Atrorosin lake comprising at least one metal, wherein at least 50-95% of the lake remains dispersed in water-based solution.

The invention solves the problem as Atrorosin lakes have increased stability matrices with high water activity and is less prone to photooxidation compared to Atrorosin dyes. In particular, the lakes provided by the present invention exhibit improvements regarding dispersibility in liquids.

In a second aspect the present invention provides a method of producing the pigments, Atrorosin lakes, comprising the steps of:
a) Priming the metal(s) to be amphoteric by making hydroxide/oxide metal without use of NaCO₃
b) Coupling Atrorosin dye to metal(s) ions by lowering the pH slowly to between 3.5-4
c) Phase separation of precipitate and liquid e.g. by filtration or (spray) drying
d) Optionally washing the lake material prior to step e)
e) Reducing particle size, e.g. by milling of the Atrorosin lake with a stabilizing agent added.

Additionally, the invention provides a method of making Atrorosin-lakes supplemented with solid hydroxy ions from alkaline earth metals such as beryllium (Be), calcium (Ca), magnesium (Mg), strontium (Sr) or barium (Ba). This supplement can alter appearance and physiochemical attributes of the Atrorosin lakes. Another benefit will be reduced aluminum which may be replaced with earth metal. This would be of interest as especially in foods, aluminium can be a potential health hazard, whereas i.e. calcium and magnesium are seen to have health benefits.

Hence, in an embodiment prior to lowering of pH in step b), an alkaline earth metal is added.

Additionally, the invention provides a kit of parts for coloring a composition, wherein the kit comprises at least one Atrorosin lake, at least one stabilizing agent wherein the pigment is supplied in a container, wherein the composition is selected from among a food, a non-food product and a cosmetic.

### BRIEF DESCRIPTION OF THE FIGURES

FIG. 1: a) Previous protocol AtrorosinE lake, dispersed in water-based fondant, b) AtrorosinE lake made by the improved protocol of the invention. AtrorosinE Lake stabilized by gum Arabic, dispersed in water-based fondant.

### DETAILED DESCRIPTION OF THE INVENTION

### Terms

Colorant: A colored substance (molecules) that is either a dye or a pigment.
Dye: Dyes are colored substances (molecules) that are soluble in the liquid/medium which they are mixed into. In general, dyes are soluble in water.
Pigment: Pigments are colored substances (molecules) that do not dissolve in liquid/medium with which they are mixed. In general, pigments are insoluble in water.
Atrorosin: A colorant having the chemical formula CQ3HQ406NR, where NR is a compound containing a primary amine, such as an amino acid, and the configuration of the double bond between carbon 2 and 3 is cis (see e.g. WO2022/008503).
Lake/Laq: Pigment where dyes are fixed to a metallic salt, rendering it insoluble.
Hue angle: In the CIEL*a*b* color system, where L* is a measure of lightness, a* is a measure of red and green (negative indicates green, positive indicates red, and b* is a measure of blue and yellow (negative indicate blue and positive indicate yellow), hue angle is a way to describe the color tone. Red colors will have a hue angle between 20 and 40. At 40 and above orange will be seen in the hue, and below 20 the color will have a magenta look.
Chroma: Chroma is the saturation of color. Colors with a high chroma are brilliant colors while colors with a low chroma are dull (pastel).
Tinctorial strength: Tinctorial strength (T*) is a measure of potency or color intensity and involves both color saturation and (chroma) and the brightness (L*). Tinctorial strength can be calculated as:

$${T}^{*} = ( 100 - {L}^{*} ) + {C}^{*}$$

The method comprises of steps in preparing Atrorosin lakes, where the Atrorosin dye is coupled to a metal. The metal can also be supplemented with an alkaline earth metal to further change the appearance and attributes.

In an embodiment the method of producing the pigments, Atrorosin lakes, comprises the steps of:
a) Priming the metal(s) to be amphoteric by making hydroxide/oxide metal without use of NaCO₃
b) Coupling Atrorosin dye to metal(s) ions by lowering the pH slowly to between 3.5-4
c) Phase separation of precipitate and liquid e.g. by filtration or (spray) drying
d) Optionally washing the lake material prior to step e)
e) Reducing particle size, e.g. by milling of the Atrorosin lake with a stabilizing agent added.

In another embodiment the method of producing the pigments, Atrorosin lakes, comprises the steps of:
a) Priming the metal(s) to be amphoteric by making hydroxide/oxide metal without use of NaCO₃
b) Coupling Atrorosin dye to metal(s) ions by lowering the pH slowly to between 3.5-4.0
c) Phase separation of precipitate and liquid e.g. by filtration or (spray) drying
d) Optionally washing the lake material prior to step e)
e) Reducing particle size, e.g. by milling of the Atrorosin lake with a stabilizing agent added.

Starting from AtrorosinE salt (AtrE) as produced via the method described in WO2022/008503. Amphoteric metal, such as aluminum hydroxide (Al(OH)₃, is prepared by dissolving aluminum potassium sulfate dodecahydrate in 0.5 M solution and adjusting the pH to 10 with 1M NaOH or KOH and heating it to 80° C. The aluminum hydroxide is coupled to AtrorosinE by mixing in molar ratios of between 1:1 and 1:3 AtrE:Al(OH)₃ and slowly under stirring lower the pH to 3.5 with 5 M HCl. This slurry is then incubated at 50° C. for 1 hour before setting it at room temperature to cool. By lowering pH to 3.5-4 a covalent and irreversible complex of metal and dye yields a non-soluble, odorless, and tasteless pigment. After cooling, the pigment can be separated from the liquid by either filtration on to a filter paper or by spray drying resulting in a dry powder. This powder can then be washed to remove remaining uncoupled Atrorosin or metal. The lake pigment can either be used as is to impart color into e.g., paints or the pigment can be formulated with a stabilizing agent such as gum arabicum. Formulation is done by mixing the stabilizing agent e.g. 25% solution in a 2:1 ratio with Atrorosin lake such as AtrorosinE lake, before wet milling with metal or ceramic beads. Surprisingly, the resulting pigments has particle sizes small enough to compete with the Brownian movement of an aqueous solution, yielding a surprisingly opaque aqueous solution where at least 50% of the lake will remain dispersed.

The lake made by the previous method described in example 8 of WO2022/008503, includes the use of sodium carbonate, which was used as a caustic to prepare aluminum hydroxide however sodium hydroxide is also used, and sodium carbonate is therefore rendered unnecessary. Omitting sodium carbonate also simplifies the process as there will be less impurities to remove in the washing step giving a higher tinctorial strength and there is no CO₂ released in the complexation step reducing the foaming to a minimum.

In the coupling step, it is important that the pH does not drop below pH 3.5 as at even lower pH values, the AtrorosinE will begin to precipitate and consequently complexation of dye and metal will fail. The yielding lake will appear black or burnt and will not impart color and cannot be dispersible but will more act as big cohesive aggregates that cannot be milled properly.

To make lakes with a high tinctorial strength and a strong chroma (saturation of color), it is also necessary to keep the molar ratio of AtrorosinE:aluminum hydroxide to be between 1:1 to 1:3 in the coupling step. At this molar ratio, all the AtrorosinE will bind to aluminum, leaving almost clear liquid after separation of the pigment, but without the excess aluminum hydroxide forming salts with the remaining anions which co-precipitate with the pigment. Lowering the ratios to be between 1:1 and 1:3, will reduce the amounts of starting material needed, and thus reduce the of aluminum potassium sulfate dodecahydrate, and further decrease the cleaning steps necessary to wash away unwanted co-precipitants. In example 8 of WO2022/008503 a ratio of 1:6 AtrorosinE:Aluminum hydroxide is used and as seen in the examples of this patent it is a more dull color with lower tinctorial strength. Example 4 of the present application shows that lakes made by the new improved protocol has a tinctorial strength which is about 1.5× higher compared to the previous protocol, and a chroma 2× higher with this new improved protocol, when tested in a fondant base.

When coupling of Atrorosin is supplemented with earth metals it is possible to alter the hue angle of the dispersed lake. Another benefit of supplementation with earth metals such as calcium and magnesium are the ability to reduce the amount of aluminum in the lake. This is especially of interest in food applications, as aluminum can be a potential health hazard, whereas i.e. calcium and magnesium are seen to have health benefits.

The present invention provides a novel method of making Atrorosin lakes that are more brilliant in color, more easily and evenly dispersed in solutions, and more color intense than lakes made by previous methods.

In some embodiments, the present invention provides an Atrorosin lake comprising at least one metal, and wherein at least 50-95% of the lake remains dispersed in water-based solution.

Dispersion percentage can be calculated by measuring the absorbance at 515 nm of a water-based solution with AtrorosinE lake, at the time of mixing (t0) and 24 hours later (t1).

$$D = \frac{{C}_{t 1}}{{C}_{t 0}} * 100$$

Measurements at 515 nm will measure red color, and aggregates will during 24 hours precipitate and the color intensity of sample measured at 515 nm will therefore decrease.

The Atrorosin lakes produced by this method will surprisingly, when its particle size is reduced significantly by ball milling with a stabilizing agent, compete with the Brownian movement and remain dispersed in aqueous solutions. Depending on how small the particle sizes is, the solutions can either be translucent or opaque. Without fine milling of the lake powder, but only using the mortar and pestle method for reducing particle size, the particle size of the lake will be larger and lake particles will agglomerate and precipitate in aqueous solutions.

In some embodiments, the present invention provides an Atrorosin lake according to the above embodiment, wherein the ratio of Atrorosin to metal is between 1:1 to 1:3 on a molar basis.

The ratio of Atrorosin to metal, will optimally be between 1:1-1:3 on a molar basis to make brilliant colors (with high chroma meaning high saturation) and a high tinctorial strength when milled. When the ratio is increased excess metal will form salts with the remaining anions (Cl, SO₄, other impurities from the Atrorosin (such as citrate)). These salts will dilute the color strength and the chroma making them dull.

In some embodiments, the invention provides Atrorosin lake according to the above embodiments, wherein the metal is selected from Aluminum, Iron, Copper, Nickel, Cobalt or Zinc.

To be able to reduce the particle size, it is necessary to mill the lake powder in the presence of a stabilizing agent to remove the remaining trapped air between the particles. The stabilizing agent acts so the grinding/milling doesn't overheat and burn the molecules. Milling without a stabilizing agent will be less efficient in reducing particle size of the lake, and thus be provide lake particles that are less efficiently and less permanently dispersed in liquid solutions, than if a stabilizing agent had been added during the milling process. Suitable stabilizing agents are in non-limiting example any one of gum arabicum, and OSA-starches have been tested as they are food grade. Others are vegetable oils, or even water.

Accordingly, the invention provides an Atrorosin lake according to any of the above embodiments, further comprising a stabilizing agent. In further embodiments, an Atrorosin lake is provided according to the previous embodiment, wherein the stabilizing agent is any of gum arabicum or Octenyl succinic anhydride (OSA)-modified waxy starches, or vegetable oils or water. In further embodiments, the Atrorosin lake the previous embodiments, comprises a stabilizing agent, wherein the ratio of stabilizing agent to Atrorosin lake is from 10:1 to 1:10 by weight.

The present inventors have surprisingly found that supplementation of the lake with alkaline earth metals can change the hue (color tone) and chroma of the preceding lake. Further, the amount of other metals such as e.g. aluminium bound to Atrorosin such as AtrorosinE may be reduced and the aluminium may be exchanged with the alkaline earth metals. This would be of interest especially in foods, as aluminium can be a potential health hazard, whereas i.e. calcium and magnesium are seen to have health benefits.

Accordingly, the present invention provided a lake according to any of the previous embodiments, wherein the lake further comprises at least one alkaline earth metal. In some such embodiments, the alkaline earth metal is any of beryllium (Be), magnesium (Mg), calcium (Ca), strontium (Sr), and barium (Ba). In preferred embodiments, the alkaline earth metal is calcium or magnesium.

In some embodiments, the Atrorosin lake according to any one of the preceding embodiments is made with AtrorosinE.

The lakes of the present invention may have different forms when sold, such as in non-limiting example in the form of a powder, or dispersed in liquid solution, such as in non-limiting example in an aqueous or oil-based solution. Accordingly, in some embodiments, the invention provides an Atrorosin lake according to any of the above embodiments, wherein the lake is in the form of a powder, dispersed in an aqueous or oil based or other liquid solution. In some embodiments, the invention provides an aqueous, oil based or other liquid solution comprising a dispersed lake according to any one of the previous embodiments.

The Atrorosin lakes provided by the present invention are brighter and more intense colored than those made by previous yet unpublished method. Accordingly, the invention provides the Atrorosin lake according to any of the previous embodiments, wherein the tinctorial strength is at least 50 and the chroma is at least 20 when measured in water-based fondant as seen e.g. example 4.

The previous and yet unpublished method of PCT/EP2021/068647 for making Atrorosin lake had a problem of providing pastel colors, and large amounts of impurities which may precipitate. Thus, the present invention provides an improved method for making Atrorosin lake wherein the amphoteric metal is made without use of NaCO₃.

The reason for removing the NaCO₃ step from the previous method is that its presence causes more impurities and is unnecessary as inventors have found that the amphoteric metal may be made with NaOH alone. The pH is lowered slowly as to avoid precipitation of both Atrorosin such as AtrorosinE but also the metals into metallic salts. In some embodiments, the invention provides a method of making an amphoteric metal by use of NaOH, without NaCO₃, such as a method of making the lake according to the invention, wherein the amphoteric metal is made without use of NaCO₃.

The invention provides in some embodiments a method for producing the Atrorosin(E) lake according to any of the previous embodiments, comprising the steps of
a) Priming the metal(s) to be amphoteric by making hydroxide/oxide metal
b) Coupling Atrorosin dye to metal(s) ions by lowering the pH slowly to between 3.5-4
c) Phase separation of precipitate and liquid e.g. by filtration or (spray) drying
d) Optionally washing the lake material prior to reducing particle size.

And in a further embodiment, the method of the previous embodiment, wherein step a) is without use of NaCO₃.

As described above, the inventors have found that having a small particle size of the lake powder is very important for its ability to be dispersed permanently in liquids, but also impacts on the brightness of the color of the lake, and on the intensity of the color. Therefore, in some embodiments the invention provides a method of making an Atrorosin lake according to the above embodiment, further comprising the step
e) Reducing particle size, e.g. by milling of the Atrorosin lake

In further embodiments, the invention provides the method according to the previous embodiments, wherein step e) includes adding a stabilizing agent. And in a further embodiment, the stabilizing agent is selected among any of gum arabicum, Octenyl succinic anhydride (OSA)-modified waxy starches, vegetable oils and water.

In some embodiments, the invention provides the method according to any of the previous embodiments, wherein the particle size of step e) is reduced to less than 20 μm, such as less than 10 μm, such as less than 2 μm. In some such embodiments, at least 90% of particles made in the milling step (e) are reduced to less than 20 μm, such as less than 10 μm, such as less than 2 μm. In some such embodiments, at least 95%, such as at least 96%, such as at least 97%, such as at least 98%, such as at least 99% of particles made in the milling step (e) are reduced to less than 20 μm, such as less than 10 μm, such as less than 2 μm.

In some embodiments, the invention provides a composition comprising the lake according to any of the previous embodiments or the lake made by the process according to any of the previous embodiments.

Lakes according to the present invention, such as Atrorosin lakes, such as AtrorosinE lakes, are interesting for colouring of a long list of products, including any of Pet food, Coating inks, Paints, Stain such as wood stain, Cosmetics, Food products, Consumer goods, plastic materials, or Food packaging.

Accordingly, the invention in some embodiments relates to use of a lake according to any the previous embodiments, the lake made by the process according to any of the previous embodiments, or the composition according to the previous embodiment, for making a product selected from the list of a Pet food, Coating inks, Paints, Stain such as wood stain, Cosmetics, Food products, Consumer goods, plastic materials, or Food packaging.

In some embodiments, the invention provides a product comprising or coloured by a lake according to any of the previous embodiments or comprising or coloured by the lake made by the process according to any of the previous embodiments or comprising or coloured by the composition according any of the previous embodiments. In further embodiments, the product provided in the previous embodiment is any one of a Pet food, Coating inks, Paints, Stain such as wood stain, Cosmetics, Food products, Consumer goods, plastic materials, or Food packaging.

In some embodiments, the invention provides a kit comprising the lake according to any of the previous embodiments for use in making any one of a Pet food, Coating inks, Paints, Stain such as wood stain, Cosmetics, Food products, Consumer goods, plastic materials, or Food packaging.

### ITEMS

1. An Atrorosin lake comprising at least one metal, wherein at least 50-95% of the lake remains dispersed in water-based solution.
2. The Atrorosin lake according to item 1, wherein the ratio of Atrorosin to metal is between 1:1 to 1:3 on a molar basis.
3. The Atrorosin lake according to items 1 and 2, wherein the metal is selected from Aluminum, Iron, Copper, Nickel, Cobalt or Zinc.
4. The Atrorosin lake according to items 1 and 2, wherein the metal is selected from Aluminum, Copper, Nickel, Cobalt or Zinc.
4. An Atrorosin lake according to any of items 1 to 3, further comprising a stabilizing agent.
5. The Atrorosin lake according to item 4, wherein the stabilizing agent is gum arabicum, Octenyl succinic anhydride (OSA)-modified waxy starches, vegetable oils or water.
6. The Atrorosin lake according to any of items 4 or 5, wherein the ratio of stabilizing agent solution to Atrorosin lake is from 2:1 to 1:10 by weight.
7. An Atrorosin lake according to any of items 1-6, wherein the lake further comprises at least one alkaline earth metal.
8. The Atrorosin lake according to item 7, wherein the alkaline earth metal is any of beryllium (Be), magnesium (Mg), calcium (Ca), strontium (Sr), and barium (Ba).
9. The Atrorosin lake according to any of items 7-8, wherein the alkaline earth metal is magnesium (Mg) or calcium (Ca).
10. The Atrorosin lake according to any one of the preceding items, wherein the Atrorosin is AtrorosinE.
11. The Atrorosin lake according to any of items 1-10, wherein the lake is in the form of a powder, dispersed in an aqueous or oil based or other liquid solution.
12. An aqueous, oil based or other liquid solution comprising a dispersed lake according to any one of the previous items.
13. The Atrorosin lake according to any of items 1-10, wherein the tinctorial strength is at least 50 and the Chroma is at least 20.
14. A method for producing the Atrorosin(E) lake according to any of items 1-11 and 13, comprising the steps of
   a) Priming the metal(s) to be amphoteric by making hydroxide/oxide metal
   b) Coupling Atrorosin dye to metal(s) ions by lowering the pH slowly to between 3.5-4
   c) Phase separation of precipitate and liquid e.g. by filtration or (spray) drying
   d) Optionally washing the lake material prior to reducing the particle size.
15. A method for producing the Atrorosin lake according to any of items 1-11 and 13, comprising the steps of
   a) Priming the metal(s) to be amphoteric by making hydroxide/oxide metal
   b) Coupling Atrorosin dye to metal(s) ions by lowering the pH slowly to between 3.5-4.0
   c) Phase separation of precipitate and liquid e.g. by filtration or (spray) drying
   d) Optionally washing the lake material prior to reducing the particle size.
16. A method for producing the Atrorosin(E) lake according to any of items 1-11 and 13, comprising the steps of
   a) Priming the metal(s) to be amphoteric by making hydroxide/oxide metal
   b) Coupling Atrorosin dye to metal(s) ions by lowering the pH slowly to between 3.5-4.4
   c) Phase separation of precipitate and liquid e.g. by filtration or (spray) drying
   d) Optionally washing the lake material prior to reducing the particle size.
17. A method for producing the Atrorosin lake according to any of items 1-11 and 13, comprising the steps of
   a) Priming the metal(s) to be amphoteric by making hydroxide/oxide metal
   b) Coupling Atrorosin dye to metal(s) ions by lowering the pH slowly to between 3.5-4.5
   c) Phase separation of precipitate and liquid e.g. by filtration or (spray) drying
   d) Optionally washing the lake material prior to reducing the particle size.
18. The method according to any of items 14-17, wherein step a) is without use of NaCO₃.
19. The method according to any of items 14-17, further comprising the step
   e) Reducing particle size, e.g. by milling of the Atrorosin lake.
20. The method according to item 19, wherein step e) includes adding a stabilizing agent.
21. The method according to item 20, wherein the particle size of step e) is reduced so that at least 90% of particles are less than 20 μm.
22. Composition comprising the lake according to any of items 1-11 and 13, or the lake made by the process according to any of items 14-21.
23. Use of a lake according to any of items 1-11 and 13, the lake made by the process according to any of items 14-21, or the composition according to item 22, for making a product selected from the list of a Pet food, Coating inks, Paints, Stain such as wood stain, Cosmetics, Food products, Consumer goods, plastic materials, or Food packaging.
24. A product comprising or coloured by a lake according to any of items 1-11 and 13, or comprising or coloured by the lake made by the process according to any of items 14-21, or comprising or coloured by the composition according to item 22.
25. The product according to item 24, wherein the product is selected from any of a Pet food, Coating inks, Paints, Stain such as wood stain, Cosmetics, Food products, Consumer goods, plastic materials, or Food packaging.

### EXAMPLES

### Example 1: Production of AtrorosinE Salt in Small Scale

This example is example 1 of WO2022/008503

5 L of fermentation broth (produced according to the methods of WO2018206590), containing approximately AU₄₉₀ of 12 of Atrorosin-E pigment, was filtered through miracloth to remove biomass and other macro-sized constituents, resulting in 4 L of permeate containing approximately AU₄₉₀ of 10 of AtrorosinE pigment. The 4 L of permeate was further filtered through an ultrafiltration membrane with a cut-off of 10 kDa, to remove majority of proteins and peptides. The 3.5 L permeate contained approximately AU₄₉₀ of 7 of AtrorosinE pigment.

The pH of the permeate was lowered to a pH of 1.34 with addition of 60 ml of 5 M HCl. The permeate was stored at 5° C. for 24 hours with stirring. During this time, the AtrorosinE pigment precipitated. After 24 hours, the mixture was filtered through a 0.8 μm cellulose membrane. The retentate was collected and dissolved in 2 L of 20 mM citrate buffer pH 6. This resulting mixture contained approximately AU₄₉₀ of 13 of AtrorosinE pigment. The mixture was frozen prior to lyophilization. 9.5 g of resulting powder was collected and measured to have an E1% of 20.

In the context of the examples E1% is:
E1% is a comparison of tinctorial strength

Absorbance of 1% (10 g/L) of product at a specific absorbance, where this is typically the maximum absorbance wavelength in nm. Maximum absorbance wavelength can be dependent on which medium the colorant is dissolved in.

For pure colorants, the E1% absorbance relates to the molar absorptivity in the following:

$${E}_{cm}^{1 \%} = 10 * ε / M$$

### Example 2: Production of Aluminum Atrorosin Lake

Amphoteric aluminum hydroxide was prepared by mixing aluminum potassium sulfate (AlK(SO₄)₂)*12H₂O with Sodium Hydroxide (NaOH). 100 ml of AlK(SO₄)₂*12H₂O was prepared by dissolving 20.3 g into 100 ml of deionized water. The solution was heated to 80° C. and stirred to dissolve all the aluminum potassium sulfate. The aluminum hydroxide Al(OH)₃ was prepared by mixing lukewarm 100 ml AlKSO₄+150 mL 1M NaOH+0.3 ml 5M HCl to give a final pH of 10.

The aluminum AtrorosinE lake was prepared by mixing the aluminum hydroxide solution with solubilized AtrorosinE (20 g (end product from e.g. the method described in example 1) in 100 ml), and the pH adjusted to 3.5 with 5M HCl. The slurry was heated for 1 hour at 50° C. followed by overnight incubation at ambient room temperature.

After overnight phase separation, the slurry was filtered on a Whatman filter grade 50 filter. In case of superfluous pigment, the pigment was easily rinsed with water and ethanol. After filtration, the lakes were dried and weighed.

### Example 3: Alternative Production of Aluminum Atrorosin Lake

The Aluminum Atrorosin lake can also be produced by an alternative process where AlK(SO₄)₂*12H₂O (acidic) is added to the dye mix and the pH is raised to pH 3.5 for pigment formation. This process has the steps:
Starting from AtrorosinE salt (AtrE) as is dissolved in water,
Aluminum potassium sulfate dodecahydrate (has a pH of 2.3) is added in a 1:1-1:3 molar ratio of Aluminum to AtrE,
pH is increased to pH 3.0-4.5, preferably ending on pH 3.5,
Follow the rest of the protocol with the slurry binding for 1 hour etc. as described in Example 2.

In this manner the Al(OH)₃ which catalyzes making the complex of Al-AtrE is created in the presence of AtrE rather than prepared beforehand. Again the most important factor here is the pH of 3.0-4.5.

### Example 4: Milling and Dispersion of Aluminum Atrorosin Lake

One example of a stabilizing agent, gum arabicum was tested with the aluminum AtrorosinE lake from Example 2 to formulate a suitable Atrorosin lake for use in foods.

25% solution of stabilizing agent was prepared by dissolving 25 g of gum arabicum into 100 ml of deionized water.

AtrorosinE lake was added to the solution of stabilizing agent, using 10 ml 25% GA and 1 g AtrorosinE lake. The solution was stirred slowly to mix and to reduce the amount of trapped air in the solution. The dispersion was then milled by addition of 5 mm metal beads and milled in a planetary ball mill. The container was filled with one third of beads before adding the Atrorosin lake gum arabicum solution. The solution was milled in 15-minute intervals at 550 RPM.

### Example 5: pH is Critical in the Process

Amphoteric aluminum hydroxide was prepared by mixing aluminum potassium sulfate (AlK(SO₄)₂)*12H₂O with Sodium Hydroxide (NaOH). 100 ml of AlK(SO₄)₂*12H₂O was prepared by dissolving 20.3 g into 100 ml of deionized water. The solution was heated to 80° C. and stirred to dissolve all the aluminum potassium sulfate. The aluminum hydroxide Al(OH)₃ was prepared by mixing lukewarm 100 ml AlKSO₄+150 mL 1M NaOH+0.3 ml 5M HCl to give a final pH of 10.

The aluminum AtrorosinE lake was prepared by mixing the aluminum hydroxide solution with solubilized AtrorosinE (20 g (end product from e.g. the method described in example 1) in 100 ml), and the pH adjusted with 5M HCl. pH was adjusted according to Table 1 below. After overnight phase separation, the slurry was filtered on a Whatman filter grade 50 filter. In case of superfluous pigment, the pigment was easily rinsed with water and ethanol. After filtration, the lakes were dried and weighed. Milling was done according to Example 4 and a 0.1% aqueous dispersion was prepared by adding 50 mg in to 50 g of water.

TABLE 1
Calculated tinctorial strength of dispersions
based on lakes prepared at different pH.

| pH | L* | a* | b* | C* | H* | T* |
|---|---|---|---|---|---|---|
| 2.5 | 11.30 | 33.03 | 18.68 | 37.95 | 29.49 | 126.65 |
| 4.0 | 15.68 | 38.74 | 25.19 | 46.21 | 33.03 | 130.53 |
| 5.5 | 25.62 | 42.4 | 30.33 | 52.13 | 35.58 | 126.51 |
| 6.5 | 38.93 | 34.37 | 19.55 | 39.54 | 29.63 | 100.61 |
| 7.8 | 49.83 | 24.56 | 11.68 | 27.20 | 25.43 | 77.37 |

The calculated tinctorial strength of dispersions based on lakes made at different pH, show that pH is a critical parameter.

### Example 6: Dispersibility of AtrorosinE Lake Dispersion

A 0.3% solution of AtrorosinE lake dispersion from example 3 was prepared by adding 300 mg into 100 ml of demineralized water. After thorough stirring, the absorbance at 515 nm of the lakes were measured. Dispersibility was calculated by a second absorbance measurement at 515 nm of the lakes after 24 hours, when sedimentation of particles have occured (See Table 2).

$$D = \frac{{C}_{t 1}}{{C}_{t 0}} * 100$$

TABLE 2

| | t0 | t1 | D |
|---|---|---|---|
| Improved protocol AtrorosinE Lake | 0.72 | 0.61 | 84.7% |

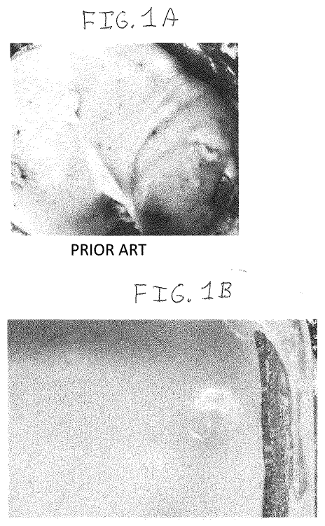

Dispersibility was also visualized photographically. AtrorosinE lake prepared by the method presented in example 8 from WO2022/008503 was milled by the same protocol as in example 2.By dispersing 150 mg of lake into a water fondant consisting of 88 g of powdered sugar into 12 ml of water. It is clear from the photos of FIG. 1 that the lake from WO2022/008503 is not dispersing evenly into the fondant, whereas the lake from Example 2 is evenly dispersed (see FIG. 1).

### Example 7: Tinctorial Strength of AtrorosinE-Lake

A 0.3% solution of AtrorosinE lake dispersion from Example 4 (Improved protocol) was prepared by adding 150 mg into 100 g of water-based fondant (88g of powdered sugar into 12 ml of water).

AtrorosinE lake prepared from the WO2022/008503 was milled by the same protocol as in example 3 (Previous protocol), and a 0.3% aqueous dispersion was prepared by adding 150 mg in to 100 g of water-based fondant.

The fondants were measured on a ColorFlex EZ from Hunterlab for their CIEL*a*b* values to compare the tinctorial strength, chroma, and hue angle (See Table 3).

TABLE 3

| | | | | | | | dE |
|---|---|---|---|---|---|---|---|
| | | | | | | | (new as |
| Batch | L* | a* | b* | C* | H* | T* | reference) |
| Improved | 63.58 | 21.26 | 8.36 | 22.84 | 21.47 | 59.26 | 0 |
| protocol | | | | | | | |
| (AtrorosinE Lake) | | | | | | | |
| Previous | 70.45 | 10.41 | 4.28 | 11.26 | 22.35 | 40.81 | 13.47 |
| protocol | | | | | | | |
| (AtrorosinE Lake) | | | | | | | |

The calculated tinctorial strength of the new improved protocol of this patent shows an 50% increase along with a chroma of 100% increase compared to the AtrorosinE lake made by the method in Example 8 of WO2022/008503. Both lakes have a similar color hue however but the chroma of the improved protocol lake is higher giving a more brilliant lake whereas the previous protocol lake is a dull color.

### Example 8: Pasteurization of Aluminum Atrorosin Lake in Soda Beverage Solution

### Model Beverage Medium Concentrate:

Sucrose: 43%
Water: 56%
Citric acid 1%

Pasteurization is a harsh treatment but also a good test for heat stability in a soda beverage. Some food colorants like betanin cannot be pasteurized so soft drinks containing these need to add the colorants after the sterilization step. Red colored beverages typically have a hue angle between 0 and 50 to appear pink to orange-reddish.

Atrorosin lake dispersion from Example 4 was mixed with 21% of concentrate with 78.97% of water and 0.01% of Atrorosin lake in a 250ml PET 1 bottle.

For comparison AtrorosinE salt and betanin was tested, and further pasteurization was performed to test the stability towards pasteurization. Concentration of AtrorosinE and Betanin were 0.045% and 0.241% respectively. Pasteurization was conducted by heating the bottles in a water bath at 90° C. for 5 minutes.

TABLE 4
Soda beverages pasteurized (90° C. for 5 minutes)

| Treatment | Sample | Avg. L | Avg. a | Avg. b | ΔE | Chroma | Hue Angle |
|---|---|---|---|---|---|---|---|
| Control | AtrorosinE | 24.5 | 51.03 | 40.83 | | 65.35 | 38.66 |
| | lake | | | | | | |
| Pastuerized | AtrorosinE | 24.89 | 51.16 | 41.22 | 0.57 | 65.70 | 38.86 |
| | lake | | | | | | |
| Control | AtrorosinE | 36.25 | 49.07 | 34.12 | | 59.76 | 34.81 |
| | lake | | | | | | |
| Pastuerized | AtrorosinE | 37.18 | 48.19 | 31.40 | 3.01 | 57.51 | 33.08 |
| | lake | | | | | | |
| Control | | 31.42 | 56.13 | 4.97 | | 56.35 | 5.06 |
| Pastuerized | | 45.97 | 28.85 | 30.98 | 40.41 | 42.33 | 47.04 |

As shown from Table 4, pasteurization had little effect on the AtrorosinE lake lightness (L*), chroma, and hue angle compared to AtrorosinE salt or betanin. The ΔE is a measure of color change and a value between 0-1 is normally invisible to the trained eye whereas a ΔE of 2-3.5 is medium difference obvious to the untrained eye whereas ΔE above 6 is a very big difference in color.

### Example 9: pH Stability of Atrorosin-Lake Dispersion in an Aqueous Solution

A 0.3% solution of AtrorosinE lake dispersion from example 3 was prepared by adding 300 mg into 100 ml of Mcllvaine buffer system which was adjusted to a given pH. AtrorosinE lake solutions were stirred and stored at room temperature for 24 hours.

The samples were measured on a ColorFlex EZ from Hunterlab for their CIEL*a*b* values to compare the chroma, and hue angle.

TABLE 5

| | L* | a* | b* | Chroma | Hue Angle |
|---|---|---|---|---|---|
| pH 3 | 30.43 | 51.46 | 49.49 | 71.40 | 43.88 |
| pH 5 | 29.31 | 51.62 | 47.65 | 70.25 | 42.71 |
| pH 6 | 28.73 | 51.69 | 46.52 | 69.54 | 41.99 |
| pH 7 | 29.82 | 51.56 | 47.46 | 70.08 | 42.63 |

As shown from the data in Table 5, pH has very little influence on the chroma and hue angle between pH 3 to pH 7 of AtrorosinE lake. The slight variation is due to small variation in the dosing level of AtrorosinE lake. The AtrorosinE lake of the invention is thus very stable at varying pH levels.

### Example 10: AtrorosinE Lake as a Function of Milling Time

Three samples of AtrorosinE lakes were prepared by adding 2.5 g of AtrorosinE lake pr ml 25% gum Arabic solution. Prior to milling the solution was stirred slowly to reduce the amount of trapped air. 40 metal beads (⅓ of the chamber volume) with a size of 0.5 mm was added to each sample prior to milling. Milling was conducted for 15 minutes, 30 minutes or 60 minutes, at 550 RPM.

After milling the samples were added to an aqueous solution, to a final concentration of 0.4% AtrorosinE dispersion.

The samples were measured on a ColorFlex EZ from Hunterlab for their CIEL*a*b* values to compare the tinctorial strength, chroma, and hue angle.

TABLE 6

| Milling | | | | | Hue | | dE(15 |
|---|---|---|---|---|---|---|---|
| time | L* | a* | b* | Chroma | Angle | T* | min ref) |
| 15 min | 24.3 | 45.2 | 34.9 | 57.2 | 37.7 | 132.88 | 0 |
| 30 min | 20.1 | 43.4 | 31.5 | 53.7 | 35.9 | 133.61 | 5.7 |
| 60 min | 17.6 | 41.3 | 27.5 | 49.7 | 33.7 | 132.10 | 10.8 |

As shown from the Table 6, increasing milling time lowers the L* value. This means that the samples are darker. Hue angle also decreases, making it appear redder. There is quite a visual difference between the 15 min and 60 min samples where the 60 min sample is clearly darker.

### Example 11: Production of Aluminum Atrorosin Lake Supplemented With Calcium or Magnesium

Amphoteric aluminum hydroxide was prepared by mixing aluminum potassium sulfate (AlK(SO₄)₂)*12H₂O with sodium hydroxide (NaOH). 100 ml of AlK(SO₄)₂*12H₂O was prepared by dissolving 20.3 g into 100 ml of deionized water. The solution was heated to 80° C. and stirred to dissolve all the aluminum potassium sulfate. The aluminum hydroxide Al(OH)₃ was prepared by mixing lukewarm 100 ml AlKSO₄+150 mL 1M NaOH+0.3 ml 5M HCl to give a final pH of 10.

The aluminum AtrorosinE complex was prepared by mixing the aluminum hydroxide solution with solubilized AtrorosinE (20g in 100 ml). 0.25M calcium hydroxide or 0.08 M magnesium hydroxide slurry (1 g/ml magnesium sulphate heptahydrate, adjusted to pH 12 by NaOH) was added to the solution. The solution was adjusted to pH to 3.5 with HCl 5M under continuous stirring dissolving the calcium hydroxide. The slurry was heated for 1 hour at 50° C. followed by overnight incubation at ambient room temperature.

After overnight phase separation, the slurry was filtered on a Whatman filter grade 50 filter. In case of superfluous pigment, the pigment was easily rinsed with water and ethanol. After filtration the lakes were dried and weighed. Milling was done according to example 3. 0.3% aqueous solutions of the lakes were prepared by adding 150 mg in to 100 g of water-based fondant (88 g of powdered sugar into 12 g of water).

The fondants were measured on a ColorFlex EZ from Hunterlab for their CIEL*a*b* values to compare the tinctorial strength, chroma, and hue angle.

TABLE 7

| | | | | | Hue | |
|---|---|---|---|---|---|---|
| | L* | a* | b* | Chroma | Angle | T* |
| Aluminum Lake | 27.7 | 44.8 | 30.4 | 54.2 | 34.1 | 126 |
| supplemented w Mg | | | | | | |
| Aluminum Lake | 58.3 | 16.3 | 9.2 | 18.7 | 29.6 | 60 |
| supplemented w Ca | | | | | | |

The resulting lakes demonstrated different tinctorial strength and have alternated hue angels compared to the aluminum lake of Example 7. The hue angle of both supplemented lakes has a more orange tint compared to the only aluminum lake.

1. (canceled)

2. An Atrorosin lake obtained by a method comprising:
priming a metal to be amphoteric by making a hydroxide/oxide metal without use of NaCO₃;
coupling Atrorosin dye to ions of the metal by lowering a pH slowly to a value ranging from 3.5 to 4.5;
phase separating precipitate and liquid; and
reducing particle size, wherein at least 50-95% of the Atrorosin lake remains dispersed in water-based solution, wherein the Atrorosin dye is AtrorosinE dye, and wherein a ratio of Atrorosin to the metal is between 1:1 to 1:3 on a molar basis.

3. The Atrorosin lake according to claim 2, wherein the metal is selected from the group consisting of Aluminum, Copper, Nickel, Cobalt, and Zinc.

4. The Atrorosin lake according to claim 2, further comprising a stabilizing agent.

5. The Atrorosin lake according to claim 4, wherein the stabilizing agent is selected from the group consisting of gum arabicum, Octenyl succinic anhydride (OSA)-modified waxy starches, vegetable oils, and water.

6. The Atrorosin lake according to claim 4, wherein ratio of stabilizing agent solution to Atrorosin lake 0 ranges from 10:1 to 1:10 by weight.

7. The Atrorosin lake according to claim 2, wherein the Atrorosin lake further comprises an alkaline earth metal.

8. The Atrorosin lake according to claim 7, wherein the alkaline earth metal is selected from the group of beryllium (Be), magnesium (Mg), calcium (Ca), strontium (Sr), and barium (Ba).

9. The Atrorosin lake according to claim 7, wherein the alkaline earth metal is magnesium (Mg) or calcium (Ca).

10. A solution comprising:
water; and
an Atrorosin lake dispersed in the water wherein the Atrorosin lake is obtained by a method comprising:
priming a metal to be amphoteric by making a hydroxide/oxide metal without use of NaCO₃;
coupling Atrorosin dye to ions of the metal by lowering a pH slowly to a value ranging from 3.5 to 4.5;
phase separating precipitate and liquid; and
reducing particle size, wherein at least 50-95% of the Atrorosin lake remains dispersed in water-based solution, wherein the Atrorosin dye is AtrorosinE dye, and wherein a ratio of Atrorosin to the metal is between 1:1 to 1:3 on a molar basis.

11. A method for producing an Atrorosin lake, comprising:
priming a metal to be amphoteric by making a hydroxide/oxide metal without use of NaCO₃;
coupling Atrorosin dye to ions of the metal by lowering a pH slowly to a value ranging from 3.5 to 4.5;
phase separating precipitate and liquid; and
reducing particle size.

12. The method according to claim 11, wherein the reducing particle size results in at least 90% of particles being less than 20 μm.

13. (canceled)

14. Use of an Atrorosin, wherein the Atrorosin lake is obtained by a method comprising:
priming a metal to be amphoteric by making a hydroxide/oxide metal without use of NaCO₃;
coupling Atrorosin dye to ions of the metal by lowering a pH slowly to a value ranging from 3.5 to 4.5;
phase separating precipitate and liquid; and
reducing particle size, wherein at least 50-95% of the Atrorosin lake remains dispersed in water-based solution, wherein the Atrorosin dye is AtrorosinE dye, and wherein a ratio of Atrorosin to the metal is between 1:1 to 1:3 on a molar basis, wherein the Atrorosin lake is used by:
making a product selected from the group consisting of a pet food, coating inks, paints, stain such as wood stain, cosmetics, food products, consumer goods, plastic materials, and food packaging.

15. A product comprising:
a base product; and
an Atrorosin lake combined with the base product, wherein the Atrorosin lake is obtained by a method comprising:
priming a metal to be amphoteric by making a hydroxide/oxide metal without use of NaCO₃;
coupling Atrorosin dye to ions of the metal by lowering a pH slowly to a value ranging from 3.5 to 4.5;
phase separating precipitate and liquid; and
reducing particle size, wherein at least 50-95% of the Atrorosin lake remains dispersed in water-based solution, wherein the Atrorosin dye is AtrorosinE dye, and wherein a ratio of Atrorosin to the metal is between 1:1 to 1:3 on a molar basis.

16. The Atrorosin lake according to claim 2, wherein the phase separating is performed by filtration or drying.

17. The Atrorosin lake according to claim 2, wherein the reducing particle size is performed by milling of the Atrorosin lake with a stabilizing agent added.

18. The Atrorosin lake according to claim 2, wherein Atrorosin lake material is washed prior to the reducing particle size.

19. The Atrorosin lake according to claim 2, wherein the metal is selected from the group consisting of Aluminum, Iron, Copper, Nickel, Cobalt, and Zinc.

20. The method according to claim 11, wherein the phase separating is performed by filtration or drying.

21. The Atrorosin lake according to claim 11, wherein the reducing particle size is performed by milling of the Atrorosin lake with a stabilizing agent added.

22. The Atrorosin lake according to claim 11, wherein Atrorosin lake material is washed prior to the reducing particle size.

23. The use according to claim 14, wherein the phase separating is performed by filtration or drying.

24. The use according to claim 14, wherein the reducing particle size is performed by milling of the Atrorosin lake with a stabilizing agent added.

25. The use according to claim 14, wherein Atrorosin lake material is washed prior to the reducing particle size.

26. The product according to claim 15, wherein the phase separating is performed by filtration or drying.

27. The product according to claim 15, wherein the reducing particle size is performed by milling of the Atrorosin lake with a stabilizing agent added.

28. The product according to claim 15, wherein Atrorosin lake material is washed prior to the reducing particle size.

29. The product according to claim 15, wherein the Atrorosin lake provides color to the base product.
